(19) United States
(12) Patent Application Publication
(10) Pub. No.: US 2015/0342566 A1
(43) Pub. Date: Dec. 3, 2015

(54) ULTRASONIC DIAGNOSIS APPARATUS AND PROGRAM

(71) Applicant: GE Medical Systems Global Technology Company, LLC, Waukesha, WI (US)

(72) Inventors: Arihiro MATSUMOTO, Tokyo (JP); Shunichiro TANIGAWA, Tokyo (JP)

(21) Appl. No.: 14/724,589

(22) Filed: May 28, 2015

(30) Foreign Application Priority Data
May 28, 2014 (JP) 2014-110032

(51) Int. Cl. A61B 8/08; A61B 8/00; A61B 8/14

(52) U.S. Cl. CPC A61B 8/485; A61B 8/14; A61B 8/463; A61B 8/5207

(57) ABSTRACT

An ultrasonic diagnosis apparatus comprises a processor for executing by a program: a transmission/reception control function of ultrasound; a measurement-value calculating function of calculating a measurement value regarding elasticity of the biological tissue in a portion for which elasticity is to be measured in the biological tissue based on echo signals of ultrasonic detecting pulses; and an evaluating function of evaluating an impact of an inhibiting factor intercepting propagation of the shear waves toward the portion for which elasticity is to be measured on image quality of an elasticity image based on data indicating brightness for a region rr in a B-mode image BI. The transmission/reception control function transmits the push pulse to such a position that degradation of image quality of the elasticity image by the inhibiting factor may be restrained based on an evaluation by the evaluating function.

### FIELD OF THE INVENTION

Embodiments of the present invention relate to an ultrasonic diagnosis apparatus and a program for detecting shear waves generated in biological tissue by an ultrasonic push pulse and calculating a measurement value for the elasticity of the biological tissue.

### BACKGROUND

There have been known elasticity measurement techniques of measuring the elasticity of biological tissue by transmitting an ultrasonic pulse (push pulse) having a high acoustic pressure from an ultrasonic probe to the biological tissue (for example, see Japanese Patent Application KOKAI No. 2012-100997). More specifically, a portion to be measured, i.e., a portion for which the elasticity is to be measured, is defined, shear waves generated in biological tissue by a push pulse are detected by ultrasonic detecting pulses in the portion to be measured, and the velocity of propagation of the shear waves and/or the elasticity value of the biological tissue are calculated to provide elasticity data. Then, an elasticity image having colors or the like according to the calculated value is displayed.

### SUMMARY OF THE INVENTION

In case that a bone, for example, lies in an area to which the push pulse is transmitted, the push pulse will not reach a region deeper than the bone. Moreover, in case that the ultrasonic probe is in loose or no contact with the body surface, the push pulse will not reach the inside of a subject. In such cases, no shear wave will reach the portion to be measured. Further, when a bone, a cyst, air or the like lies in a propagation path of the shear waves toward the portion to be measured, it may adversely affect propagation of the shear waves toward the portion to be measured.

Thus, when propagation of shear waves toward a portion to be measured is intercepted, it may be difficult to display an elasticity image in which the elasticity of biological tissue is more accurately reflected.

The invention in one aspect made for solving the problem described above is an ultrasonic diagnostic apparatus comprising a processor for executing by a program: a transmission control function of controlling transmission of an ultrasonic push pulse to biological tissue in a subject, transmission of a plurality of ultrasonic detecting pulses for detecting shear waves generated in said biological tissue by said push pulse, and transmission of ultrasound for producing a B-mode image for said biological tissue; a measurement-value calculating function of calculating a measurement value regarding elasticity of said biological tissue in a portion for which elasticity is to be measured in said biological tissue based on echo signals of said ultrasonic detecting pulses; an evaluating function of evaluating an impact of an inhibiting factor intercepting propagation of said shear waves toward said portion for which elasticity is to be measured on image quality of an elasticity image based on data indicating brightness for a predefined region in said B-mode image; and an image display processing function of displaying said B-mode image and said elasticity image, said apparatus being characterized in that: said transmission control function transmits said push pulse to such a position that degradation of image quality of said elasticity image by said inhibiting factor may be restrained based on an evaluation by said evaluating function.

According to embodiments of the present invention, the evaluating function evaluates an impact of an inhibiting factor intercepting propagation of the shear waves toward the portion for which elasticity is to be measured on image quality of an elasticity image based on data indicating brightness for a predefined region in a B-mode image, and the push pulse is transmitted based on the evaluation; therefore, the push pulse may be transmitted so that propagation of shear waves toward the portion to be measured would not be intercepted.

### BRIEF DESCRIPTION OF THE DRAWINGS

FIG. 1 is a block diagram showing a schematic configuration of an ultrasonic diagnostic apparatus that is an exemplary embodiment of the present invention.

FIG. 2 is a block diagram showing a configuration of an echo data processing section.

FIG. 3 is a block diagram showing a configuration of a display processing section.

FIG. 4 is a diagram showing a display section in which a B-mode image and an elasticity image are displayed.

FIG. 5 is a flow chart showing an operation of determination of a transmission position in the ultrasonic diagnosis apparatus in the first embodiment.

FIG. 6 is a diagram showing the display section in which a region of interest is defined in the B-mode image.

FIG. 7 is a diagram showing exemplary regions for which brightness information is calculated.

FIG. 8 is a diagram explaining the regions shown in FIG. 7.

FIG. 9 is a diagram explaining other exemplary regions for which brightness information is calculated.

FIG. 10 is an explanatory diagram illustrating a case in which a bone lies in an area to which a push pulse is transmitted.

FIG. 11 is an explanatory diagram illustrating a case in which a cyst lies in a propagation path of shear waves from a push pulse to a region of interest.

FIG. 12 is a flow chart showing an operation of fixing of a transmission position in the ultrasonic diagnosis apparatus in a second embodiment.

FIG. 13 is a diagram showing an exemplary region for which brightness information is calculated.

FIG. 14 is a diagram showing another exemplary region for which brightness information is calculated.

FIG. 15 is a low chart showing an operation of fixing of a transmission position in the ultrasonic diagnosis apparatus in a second variation of the second embodiment.

FIG. 16 is a diagram explaining a transmission position to which a push pulse is to be transmitted in a third variation of the second embodiment.

FIG. 17 is a diagram explaining a transmission position to which a push pulse is to be transmitted different from that in FIG. 16 in the third variation of the second embodiment.

FIG. 18 is a diagram explaining transmission of a push pulse when the region of interest is divided in the third variation of the second embodiment.

FIG. 19 is a diagram explaining transmission of a push pulse when the region of interest is divided in the third variation of the second embodiment.

FIG. 20 is a flow chart showing an operation of fixing of a beam profile in the ultrasonic diagnosis apparatus in a fourth variation of the second embodiment.

FIG. 21 is a diagram explaining modification of the width of a transmission aperture for a push pulse.

FIG. 22 is a diagram explaining determination of the width of the transmission aperture.

FIG. 23 is a diagram explaining modification of the beam direction for a push pulse.

### DETAILED DESCRIPTION

Now embodiments of the present invention will be described with reference to the accompanying drawings.

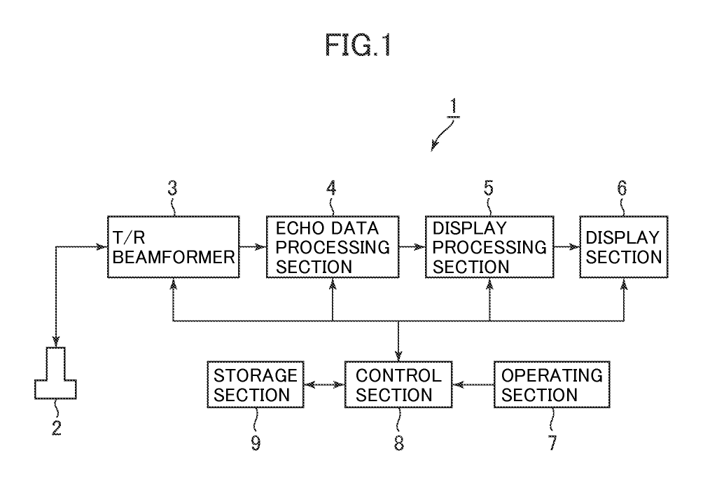

To begin with, a first embodiment will be described. An ultrasonic diagnostic apparatus 1 shown in FIG. 1 comprises an ultrasonic probe 2, a transmission/reception (T/R) beamformer 3, an echo data processing section 4, a display processing section 5, a display section 6, an operating section 7, a control section 8, and a storage section 9.

The ultrasonic probe 2 transmits ultrasound to biological tissue in a subject. By the ultrasonic probe 2, an ultrasonic pulse (push pulse) for generating shear waves in the biological tissue is transmitted. Also by the ultrasonic probe 2, ultrasonic detecting pulses for detecting shear waves generated in the biological tissue by the push pulse are transmitted and echo signals thereof are received.

Further, by the ultrasonic probe 2, ultrasonic B-mode imaging pulses for producing a B-mode image are transmitted and echo signals thereof are received.

The T/R beamformer 3 drives the ultrasonic probe 2 based on control signals from the control section 8 to transmit the several kinds of ultrasonic pulses described above with predetermined transmission parameters (transmission control function). The T/R beamformer 3 also applies signal processing such as phased addition processing to ultrasonic echo signals. The transmission beamformer 3 and control section 8 represent an exemplary embodiment of the transmission control section in the present invention. The transmission control function represents an exemplary embodiment of the transmission control function in the present invention.

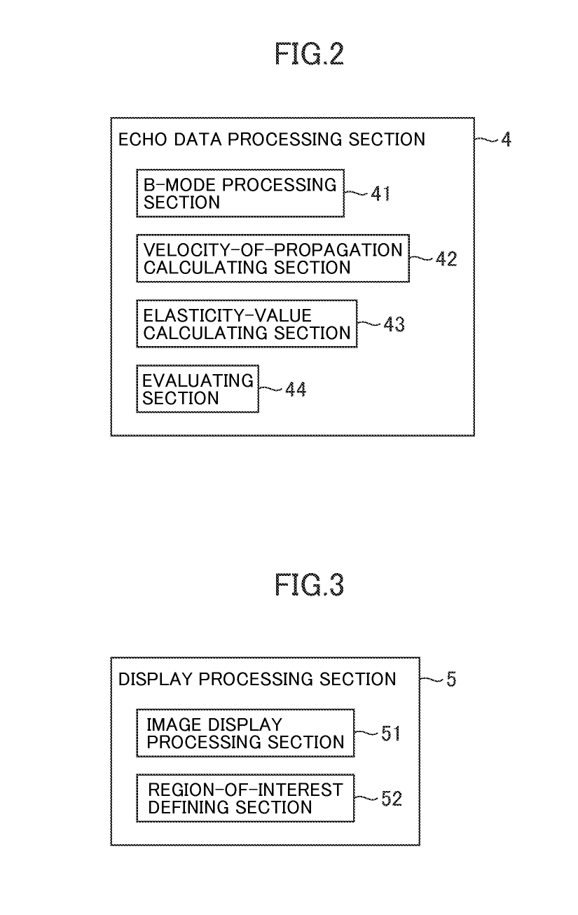

The echo data processing section 4 comprises a B-mode processing section 41, a velocity-of-propagation calculating section 42, an elasticity-value calculating section 43, and an evaluating section 44, as shown in FIG. 2. The B-mode processing section 41 applies B-mode processing such as logarithmic compression processing and envelope detection processing to echo data output from the T/R beamformer 3, and creates B-mode data.

The velocity-of-propagation calculating section 42 calculates a velocity of propagation of the shear waves based on echo data of the ultrasonic detecting pulses output from the T/R beamformer 3 (velocity-of-propagation calculating function). The elasticity-value calculating section 43 calculates an elasticity value of the biological tissue to which a push pulse is transmitted based on the velocity of propagation (elasticity-value calculating function). Details thereof will be discussed later. The velocity-of-propagation calculating function and elasticity-value calculating function represent an exemplary embodiment of the measurement-value calculating function in the present invention. The velocity of propagation and elasticity value represent an exemplary embodiment of the measurement value regarding elasticity of biological tissue in the present invention.

It should be noted that only the velocity of propagation may be calculated without necessarily calculating the elasticity value. Data of the velocity of propagation or data of the elasticity value will be referred to herein as elasticity data.

The evaluating section 44 evaluates an impact of an inhibiting factor intercepting propagation of the shear waves toward a region of interest defined in a B-mode image on image quality of the elasticity image, which will be discussed later. The evaluating section 44 evaluates an impact of the inhibiting factor on image quality of the elasticity image based on the B-mode data or B-mode image data discussed later. Details thereof will be discussed later.

The display processing section 5 comprises an image display processing section 51 and a region-of-interest defining section 52, as shown in FIG. 3. The image display processing section 51 scan-converts the B-mode data by a scan converter to create B-mode image data, based on which a B-mode image is displayed in the display section 6. The image display processing section 51 also scan-converts the elasticity data by the scan converter to create elasticity image data, based on which an elasticity image is displayed in the display section 6. The image display processing section 51 represents an exemplary embodiment of the image display processing section in the present invention. The image display processing functions described above by the image display processing section 51 represent an exemplary embodiment of the image display processing function in the present invention.

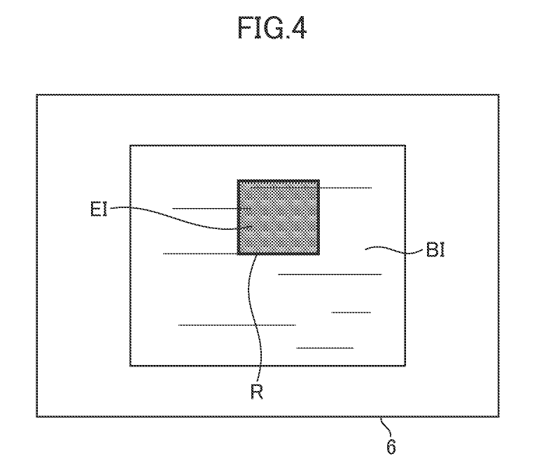

As shown in FIG. 4, the elasticity image EI is a two-dimensional image displayed within a region of interest R defined in the B-mode image BI. The elasticity image EI is a color image having colors according to the velocity of propagation or the elasticity value. The image display processing section 51 combines the B-mode image data and elasticity image data together to create combined image data, based on which an image is displayed in the display section 6. Therefore, the elasticity image EI is a semi-transparent image through which the B-mode image BI in the background is allowed to pass.

The region of interest R is defined by the region-of-interest defining section 52. More specifically, the region-of-interest defining section 52 defines the region of interest R based on an input by an operator at the operating section 7. The region of interest R is a region to/from which the ultrasonic detecting pulses are transmitted/received. The region of interest R represents an exemplary embodiment of the portion for which elasticity is to be measured in the biological tissue.

The display section 6 is an LCD (Liquid Crystal Display), an organic EL (Electro-Luminescence) display, or the like. The operating section 7 is configured to comprise a keyboard for allowing an operator to input a command and/or information, a pointing device such as a trackball, and the like, although not particularly shown.

The control section 8 is a processor such as a CPU (Central Processing Unit). The control section 8 loads thereon a program stored in the storage section 9 and controls several sections in the ultrasonic diagnostic apparatus 1. For example, the control section 8 loads thereon a program stored in the storage section 9 and executes functions of the T/R beamformer 3, echo data processing section 4, and display processing section 5 by the loaded program.

The control section 8 may execute all of the functions of the T/R beamformer 3, all of the functions of the echo data processing section 4, and all of the functions of the display processing section 5 by the program, or execute only some of the functions by the program. In case that the control section 8 executes only some of the functions, the remaining functions may be executed by hardware such as circuitry.

It should be noted that the functions of the T/R beamformer 3, echo data processing section 4, and display processing section 5 may be implemented by hardware such as circuitry.

The storage section 9 is an HDD (Hard Disk Drive), and/or a semiconductor memory such as a RAM (Random Access Memory) and/or a ROM (Read-Only Memory).

Next, an operation of the ultrasonic diagnostic apparatus 1 in the present embodiment will be described. According to the ultrasonic diagnosis apparatus 1 in the present embodiment, an elasticity image EI is displayed in a region of interest R defined in a B-mode image BI. The elasticity image EI is displayed by transmitting a push pulse and ultrasonic detecting pulses.

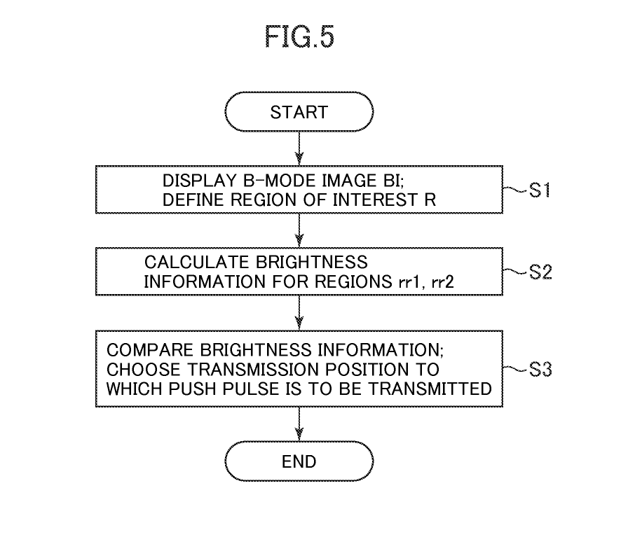

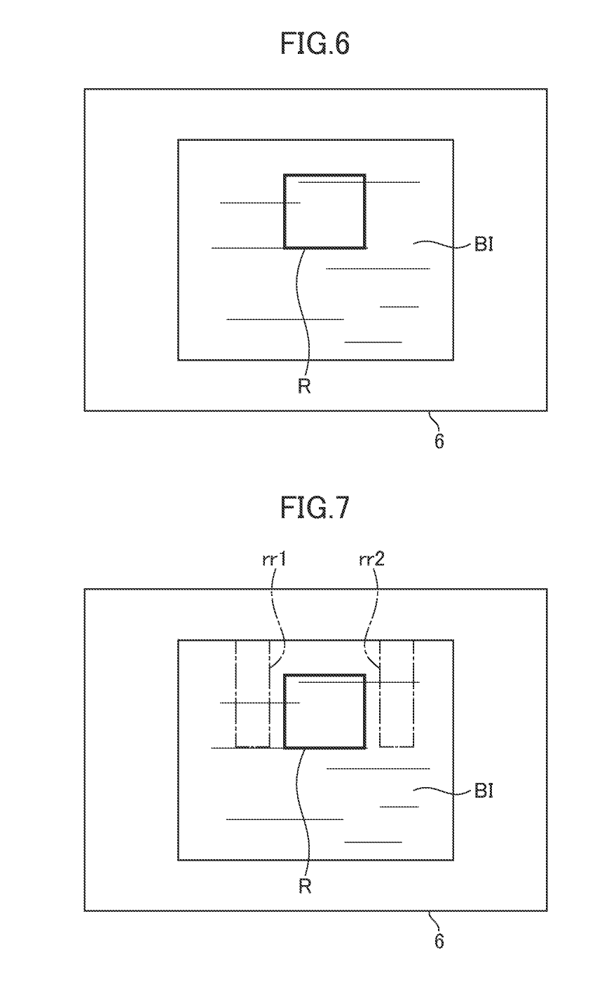

In the present embodiment, a transmission position to which a push pulse is to be transmitted is determined before displaying the elasticity image EI. The determination of a transmission position to which a push pulse is to be transmitted will now be explained based on a flow chart in FIG. 5. First, at Step S1, an operator conducts transmission/reception of ultrasound for a B mode to a subject, and displays a B-mode image BI based on echo signals, as shown in FIG. 6. The operator then defines a region of interest R in the B-mode image BI. The region of interest R is defined in an area for which an elasticity image is desired to be displayed.

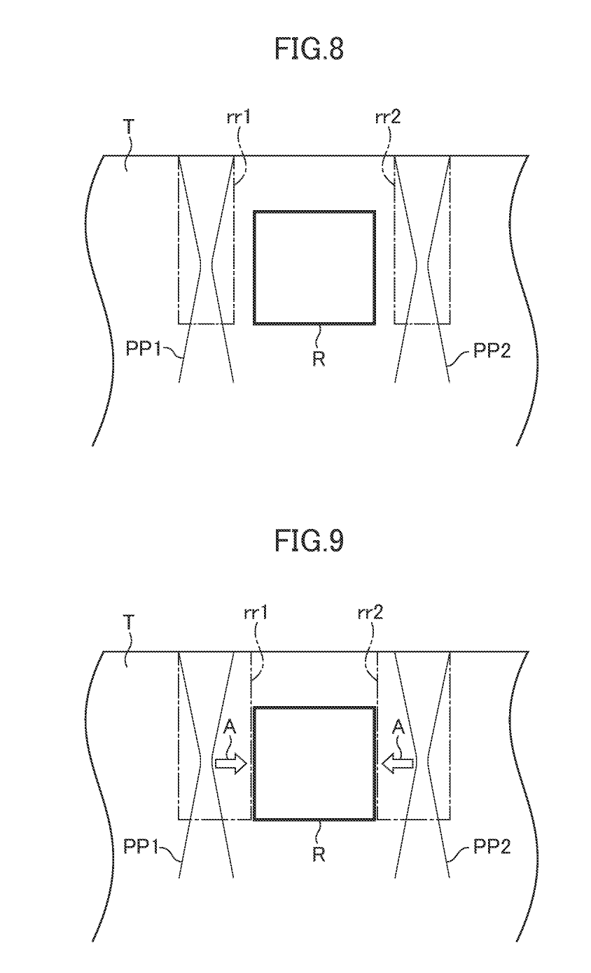

Next, at Step S2, the evaluating section calculates brightness information for regions rr1, rr2 in the B-mode image BI, as shown in FIG. 7. In the present embodiment, the brightness information is a mean brightness for each of the regions rr1, rr2. As shown in FIG. 8, the region rr1 includes an area in biological tissue T in the subject to which a push pulse PP1 is to be transmitted. The region rr2 includes an area to which a push pulse PP2 is to be transmitted.

The positions of the push pulses PP1, PP2 are determined based on a region of interest R once it has been defined. In the present embodiment, the positions of the push pulses PP1, PP2 lie on both sides of the region of interest R in a direction (transverse direction) intersecting a depth direction (acoustic line direction). The evaluating section 44 defines the region rr1 based on the transmission position to which the push pulse PP1 is to be transmitted thus determined based on the region of interest R. Likewise, the evaluating section 44 defines the region rr2 based on the transmission position to which the push pulse PP2 is to be transmitted determined based on the region of interest R. The positions of the regions rr1, rr2 also lie on both sides of the region of interest R in the transverse direction. The regions rr1, rr2 represent an exemplary embodiment of the predefined region in the present invention.

The regions rr1, rr2 may each lie at a position defined beforehand in an area to which a respective one of the push pulses PP1, PP2 is to be transmitted so that the position includes the area to which the respective one of the push pulses PP1, PP2 is to be transmitted in the biological tissue T. The positions of the regions rr1, rr2 shown in FIG. 8 are provided by way of example, and their positions are not limited thereto. For example, the regions rr1, rr2 may each lie at a position including the whole area through which the shear waves propagate from the respective one of the push pulses PP1, PP2 to the region of interest R, as shown in FIG. 9. It should be noted that the shear waves propagate from the push pulses PP1, PP2 in the transverse direction (in directions indicated by arrows A in FIG. 9). While in FIG. 8, there is a gap between each of the regions rr1, rr2 and the region of interest R in the transverse direction, no gap is present in FIG. 9 between each of the regions rr1, rr2 and the region of interest R in the transverse direction.

The evaluating section 44 may calculate a mean brightness for each of the regions rr1, rr2 based on the B-mode image data. The evaluating section 44 may also calculate a mean brightness for each of the regions rr1, rr2 based on B-mode data, which is the data before being scan-converted into B-mode image data. In other words, a mean brightness for each of the regions rr1, rr2 is a mean value of data values of the B-mode image data or of the B-mode data in each of the regions rr1, rr2. The B-mode image data and B-mode data represent an exemplary embodiment of the data indicating brightness for a predefined region in a B-mode image.

Next, at Step S3, the evaluating section 44 compare brightness information for the region rr1, i.e., the mean brightness for the region rr1, with brightness information for the region rr2, i.e., the mean brightness for the region rr2. The evaluating section 44 then chooses the transmission position to which a push pulse is to be transmitted that corresponds to one of the regions rr1, rr2 having a higher mean brightness. For example, when the mean brightness for the region rr1 is higher than that for the region rr2, the evaluating section 44 chooses the transmission position to which the push pulse PP1 is to be transmitted.

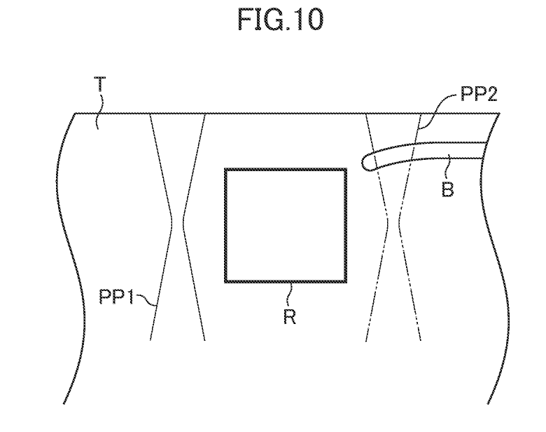

Now let us explain a reason why the transmission position to which a push pulse is to be transmitted is chosen based on the mean brightness. When an inhibiting factor intercepting propagation of ultrasound or shear waves, such as a bone, a cyst, or air, lies in an area to which the push pulse PP is transmitted and/or in a propagation path of shear waves from the push pulse PP to the region of interest R, propagation of the shear waves toward the region of interest R is intercepted. As used herein, the phrase “propagation of the shear waves toward the region of interest R is intercepted ” implies a phenomenon that no shear wave reaches the region of interest R, and a phenomenon that shear waves propagating toward the region of interest R undergo refraction or the like. For example, when a bone B lies in an area to which the push pulse PP2 is transmitted in the biological tissue T as shown in FIG. 10, the push pulse PP2 will not reach deeper than the bone B in the ultrasound acoustic line direction. Since shear waves are generated by a push pulse, no shear wave will be generated from a portion reached by no push pulse. Therefore, no shear wave reaches the region of interest R.

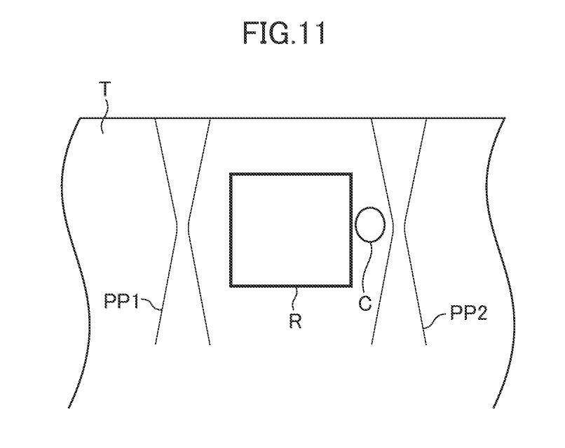

When a cyst C lies in a propagation path of shear waves from the push pulse PP to the region of interest R, as shown in FIG. 11, the shear waves undergo refraction or the like at the cyst C.

The refraction experienced by the shear waves propagating toward the region of interest R may adversely affect measurement of the velocity of propagation of the shear waves to thereby prevent production of an elasticity image in which the elasticity of biological tissue is more accurately reflected. Moreover, when no shear wave propagates toward the region of interest R as described above, an elasticity image in which the elasticity of biological tissue is more accurately reflected cannot be obtained. The inhibiting factor intercepting propagation of shear waves toward the region of interest R as used herein refers to any factor causing degradation of image quality of an elasticity image. The image quality of an elasticity image implies how accurately elasticity of biological tissue is reflected.

Furthermore, in case that a portion in a ultrasound transmission/reception plane of the ultrasonic probe 2 from which a push pulse is transmitted is in loose or no contact with the body surface of the subject, no push pulse will propagate the inside of the biological tissue. Therefore, the inhibiting factor for shear waves toward the region of interest R include the event that the ultrasonic probe 2 is in loose or no contact with the body surface.

When the push pulse does not reach deeper than a bone, B-mode imaging ultrasound also does not reach deeper than the bone, and therefore, the brightness in a portion deeper than the bone in a B-mode image in the ultrasound acoustic line direction lowers as compared with surrounding portions. Similarly, the brightness of a portion in which a cyst or air lies in the B-mode image lowers as compared with surrounding portions. When a portion in a ultrasound transmission/reception plane of the ultrasonic probe 2 from which a push pulse is transmitted is in loose or no contact with the body surface of the subject, B-mode imaging ultrasound also does not propagate the inside of the biological tissue from the loosely or non contacting portion. Therefore, the brightness in a portion below the loosely or non contacting portion in the B-mode image in the acoustic line direction lowers as compared with surrounding portions.

When the inhibiting factor is thus present, a mean brightness for the region rr1 or rr2 lowers. Therefore, the evaluating section 44 evaluates an impact of the inhibiting factor on image quality of the elasticity image by comparing the mean brightness for the region rr1 with that for the region rr2. Specifically, when a push pulse is transmitted to a position corresponding to a region having a lower mean brightness, propagation of shear waves toward the region of interest R may be probably intercepted by the inhibiting factor to degrade image quality of an elasticity image; thus, the evaluating section 44 chooses a transmission position to which a push pulse is to be transmitted corresponding to a region having a higher mean brightness. Therefore, the transmission position is such a position that degradation of image quality of the elasticity image by the inhibiting factor may be restrained.

Once a transmission position to which a push pulse is to be transmitted has been chosen by the evaluating section 44 at Step S3 described above, the push pulse is transmitted to that position. Then, transmission/reception of ultrasonic detecting pulses for detecting shear waves generated by the push pulse is performed. Based on echo signals of the ultrasonic detecting pulses, the velocity of propagation of the shear waves and the elasticity value for the biological tissue are calculated and an elasticity image EI is displayed in the region of interest R (see FIG. 4).

According to the embodiment described above, since a push pulse is transmitted to a position corresponding to one of the regions rr1, rr2 having a higher mean brightness, the push pulse may be transmitted so that propagation of shear waves toward the region of interest R would not be intercepted. Thus, an elasticity image in which the elasticity of biological tissue is more accurately reflected may be displayed.

Next, a variation of the first embodiment will be described. The brightness information calculated at Step S2 may be the degree of dispersion of brightness in each of the regions rr1, rr2. The evaluating section 44 calculates a degree of dispersion of data values of the B-mode image data or B-mode data in the region rr1 and a degree of dispersion of data values of the B-mode image data or the B-mode data in the region rr2. The degree of dispersion is a standard deviation, a variance or a coefficient of variation.

At Step S3 described above, the evaluating section 44 compares the degree of dispersion of brightness in the region rr1 with that in the region rr2. The evaluating section 44 chooses a transmission position to which a push pulse is to be transmitted corresponding to one of the regions rr1 and rr2 having a lower degree of dispersion of brightness.

Now let us explain a reason why the transmission position to which a push pulse is to be transmitted corresponding to one of the regions rr1 and rr2 having a lower degree of dispersion of brightness is chosen. When an inhibiting factor intercepting propagation of shear waves is present and a portion with lowered brightness exists in the regions rr1, rr2 in a B-mode image, the degree of dispersion of brightness increases in the regions rr1, rr2. Accordingly, the evaluating section 44 chooses a transmission position to which a push pulse is to be transmitted corresponding to a region having a lower degree of dispersion of brightness.

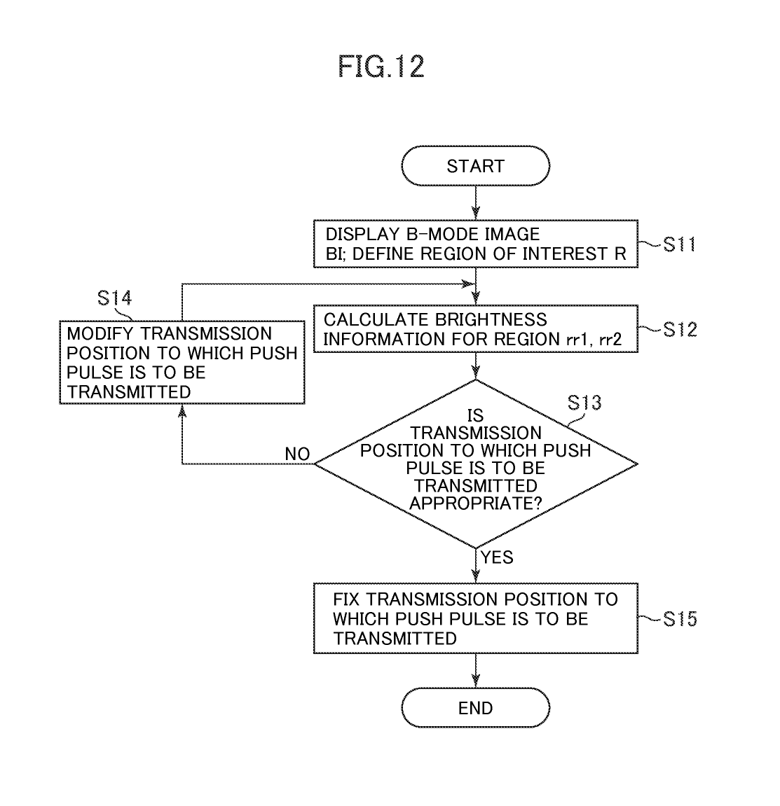

Next, a second embodiment will be described. Since the configuration of the ultrasonic diagnosis apparatus in the present embodiment is identical to that of the first embodiment, determination of a transmission position to which a push pulse is to be transmitted will be described based on a flow chart in FIG. 12.

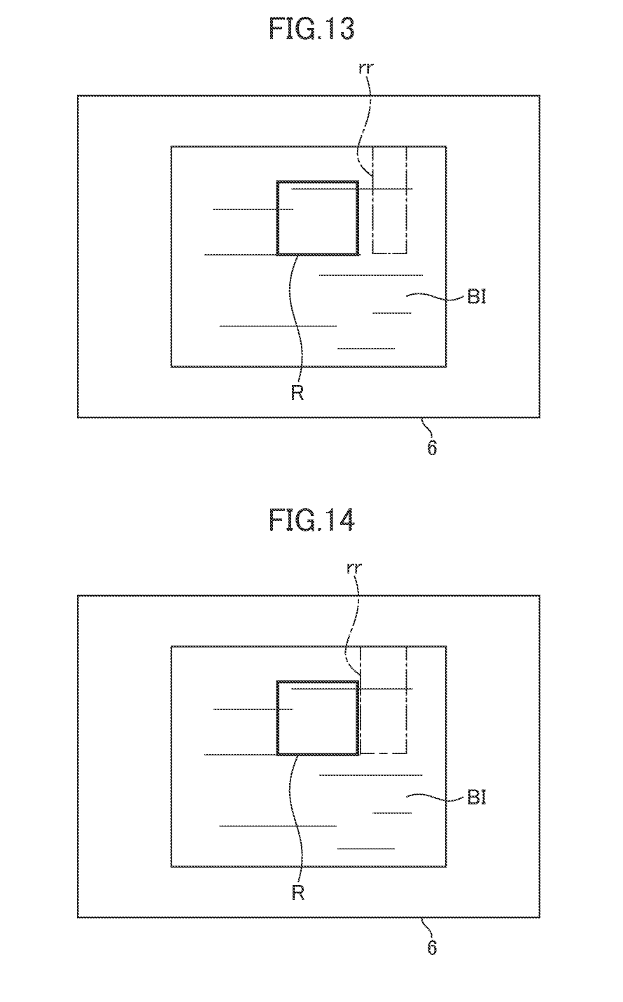

First, at Step S11, a B-mode image BI is displayed and a region of interest R is defined, as in Step S1 described earlier. Next, at Step S12, the evaluating section 44 calculates brightness information for a region rr in the B-mode image BI shown in FIG. 13. The brightness information is a mean brightness for the region rr. Similarly to the regions rr1, rr2, the region rr includes an area to which a push pulse is to be transmitted. The region rr shown in FIG. 13 is provided by way of example, and the region rr may be defined so that no gap is present between the region rr and region of interest R, as shown in FIG. 14.

The mean brightness for the region rr is a mean value of data values of B-mode image data or B-mode data in the region rr again in the present embodiment.

Next, at Step S13, the evaluating section 44 evaluates an impact of the inhibiting factor on image quality of an elasticity image by deciding whether the brightness information for the region rr meets predefined criteria or not to evaluate whether or not the transmission position to which a push pulse is to be transmitted corresponding to the region rr is appropriate. In particular, the evaluating section 44 compares a mean brightness Br in the region rr with a threshold Brth, and decides whether Br>Brth (the predefined criteria) is satisfied or not. The threshold Brth is set to such a value that degradation of image quality of the elasticity image by the inhibiting factor probably cannot be restrained because of a low mean brightness for the region rr.

When Br>Brth, the evaluating section 44 evaluates that degradation of image quality of the elasticity image by the inhibiting factor can be restrained and the transmission position to which a push pulse is to be transmitted is appropriate (“YES” at Step S13). On the other hand, when Br<=Brth, the evaluating section 44 evaluates that degradation of image quality of the elasticity image by the inhibiting factor cannot be restrained and the transmission position to which a push pulse is to be transmitted is not appropriate (“NO” at Step S13).

When the transmission position to which a push pulse is to be transmitted is decided not to be appropriate at Step S13, the flow goes to processing at Step S14. At Step S14, the control section 44 modifies the transmission position to which a push pulse is to be transmitted. The evaluating section 44 newly defines a region rr according to the modified transmission position to which a push pulse is to be transmitted. Specifically, the evaluating section 44 newly defines a region rr including an area to which a modified push pulse is to be transmitted.

Once the region rr has been newly defined at Step S14, the flow goes back to processing of Step S12, and a mean brightness is calculated for the newly defined region rr.

On the other hand, the transmission position to which a push pulse is to be transmitted is decided to be appropriate at Step S13, the evaluating section 44 fixes the transmission position decided to be appropriate at Step S13 as transmission position for a push pulse transmitted to the subject at Step S15. At Step S15, once the transmission position to which a push pulse is to be transmitted has been fixed, a push pulse is transmitted to that position and transmission/reception of the ultrasonic detecting pulses is performed, as in Step S4 described earlier. The elasticity image EI is then displayed.

According to the embodiment described above, when the condition Br>Brth is not satisfied, the transmission position to which a push pulse is to be transmitted is modified at Step S14. Then, a push pulse is transmitted to the transmission position corresponding to the region rr satisfying the condition Brth<Br, and thus, the push pulse may be transmitted so that propagation of shear waves toward the region of interest R would not be intercepted. Thus, an elasticity image in which the elasticity of biological tissue is more accurately reflected may be displayed.

Next, a variation of the second embodiment will be described. Similarly to the variation of the first embodiment, the brightness information calculated at Step S12 described above may be the degree of dispersion of brightness in the region rr. In this case, the evaluating section 44 compares the degree of dispersion D in the region rr with a threshold Dth and decides whether D<Dth or not at Step S13. The presence of an area having a low brightness in the region rr results in a higher degree of dispersion. Accordingly, the threshold Dth is set to such a value that degradation of image quality of an elasticity image by the inhibiting factor probably cannot be restrained because an area having a low brightness is present in the region rr.

When D<Dth, the evaluating section 44 decides that the transmission position to which a push pulse is to be transmitted is appropriate. On the other hand, when D>=Dth, the evaluating section 44 decides that the transmission position to which a push pulse is to be transmitted is not appropriate.

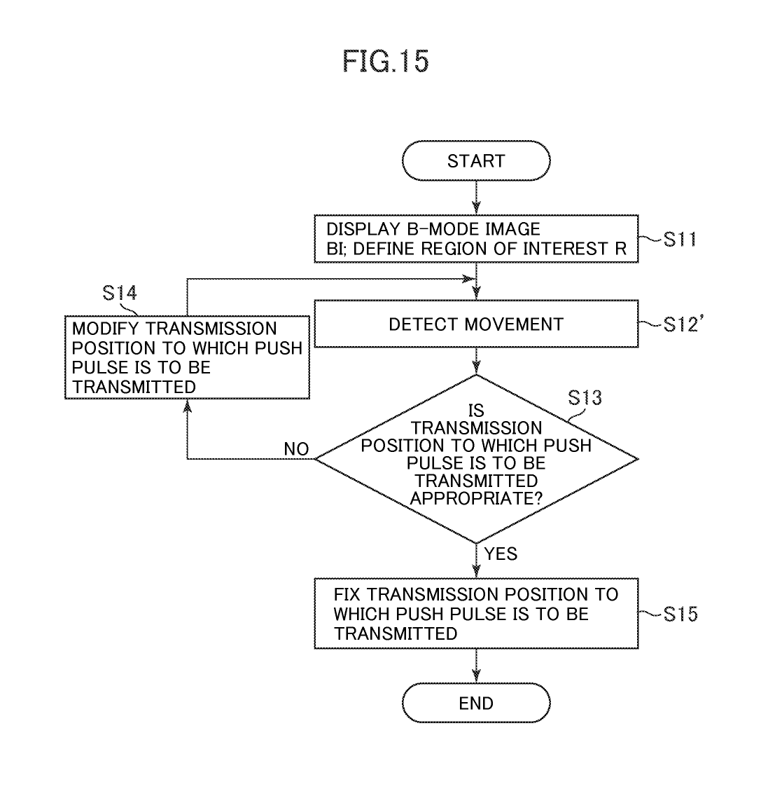

Next, a second variation will be described based on a flow chart in FIG. 15. In the flow chart in FIG. 15, explanation of the same processing as that in FIG. 12 will be omitted. In the flow chart in FIG. 15, the evaluating section 44 detects movement of a portion corresponding to the region rr in the biological tissue based on B-mode image data or B-mode data at Step S12′. The evaluating section 44 performs the detection of movement by performing a correlation calculation, for example, on B-mode image data or B-mode data in two temporally different frames for the same cross-sectional plane.

In case that a portion corresponding to the region rr in the biological tissue is moving due to heartbeats, etc. and a push pulse is transmitted to such a region rr, the movement of the biological tissue affects propagation of shear waves. Consequently, image quality of the elasticity image may be degraded. Accordingly, in case that any movement is detected at Step S12′, the evaluating section 44 decides at Step S13 that the transmission position to which a push pulse is to be transmitted is not an appropriate position for restraining degradation of image quality of the elasticity image. On the other hand, in case that no movement is detected at Step S 12′, the evaluating section 44 decides that the transmission position to which a push pulse is to be transmitted is appropriate at Step S13.

Next, a third variation will be described. In the third variation, a plurality of the push pulses are transmitted to different positions, and elasticity image data created based on echo signals of detecting pulses corresponding to each push pulse is summed up to create elasticity image data in one frame.

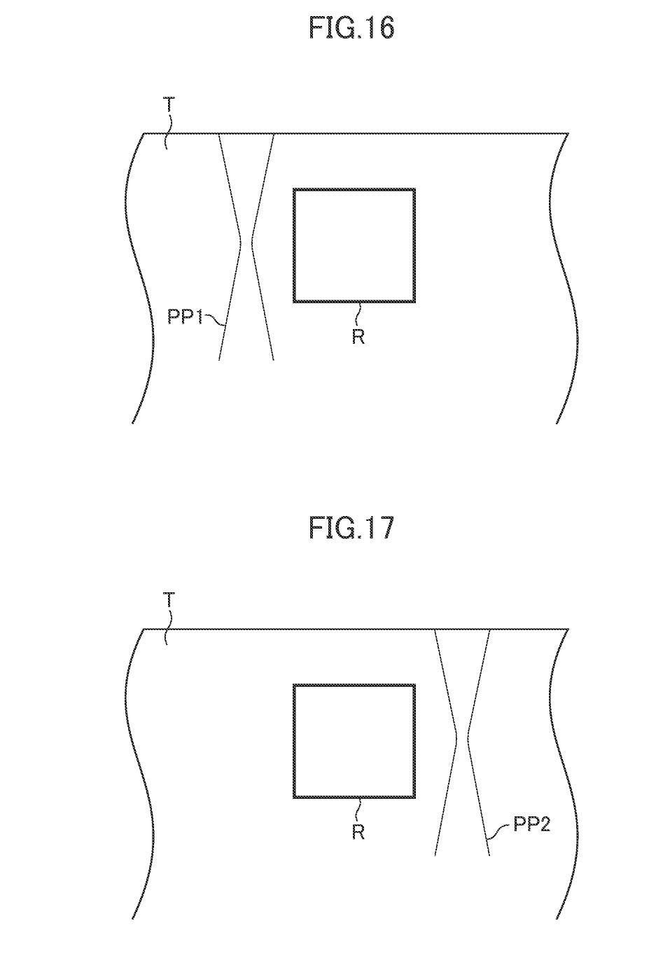

For example, after a push pulse PP1 has been transmitted as shown in FIG. 16, transmission/reception of ultrasonic detecting pulses (not shown) for detecting shear waves generated by the push pulse PP1 is performed. Thereafter, as shown in FIG. 17, a push pulse PP2 is transmitted and transmission/reception of ultrasonic detecting pulses (not shown) for detecting shear waves generated by the push pulse PP2 is performed. Then, elasticity image data in one frame is created by summing up elasticity image data in one frame created based on echo signals of the ultrasonic detecting pulses corresponding to the push pulse PP1 and elasticity image data in one frame created based on echo signals of the ultrasonic detecting pulses corresponding to the push pulse PP2.

In the third variation as described above, fixing of the transmission positions for the push pulses PP1, PP2 is achieved as in the flow chart in FIG. 12 or 15 described above. Specifically, the processing in the flow chart in FIG. 12 or 15 is applied to each of the push pulses PP1, PP2 to fix their transmission positions.

In case that the transmission position for one of the push pulses PP1, PP2 is decided not to be appropriate at Step S13 described above, transmission of the push pulse to the position decided not to be appropriate may be omitted. In this case, a push pulse for which the transmission position is decided to be appropriate is transmitted and transmission/reception of ultrasonic detecting pulses for shear waves generated by the push pulse is performed for one frame to create elasticity image data in one frame. Moreover, transmission/reception of ultrasonic detecting pulses corresponding to a push pulse for which the transmission position is decided to be appropriate may be performed for two frames, and elasticity image data in the two frames may be summed up to create elasticity image data in one frame.

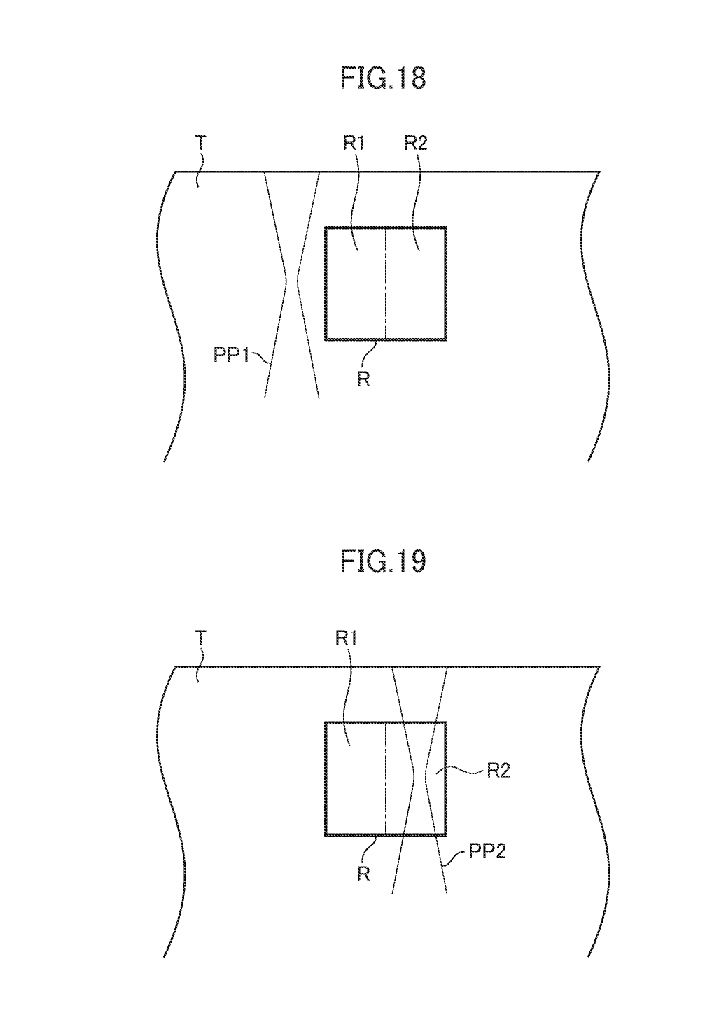

Alternatively, as shown in FIGS. 18 and 19, it is possible to divide the region of interest R into a first region of interest R1 and a second region of interest R2, and transmit the push pulses PP1, PP2 to both sides of each of the first and second regions of interest R1, R2. FIGS. 18 and 19 show a case in which the push pulses PP1, PP2 are transmitted to both sides of the first region of interest R1.

In this case, elasticity image data for the region of interest R in one frame is created based on elasticity image data for the first region of interest R1 in one frame created based on echo signals of ultrasonic detecting pulses corresponding to each of the push pulses PP1, PP2 transmitted to both sides of the first region of interest R1 and elasticity image data for the second region of interest R2 in one frame created based on echo signals of ultrasonic detecting pulses corresponding to each of the push pulses PP1, PP2 transmitted to both sides of the second region of interest R2.

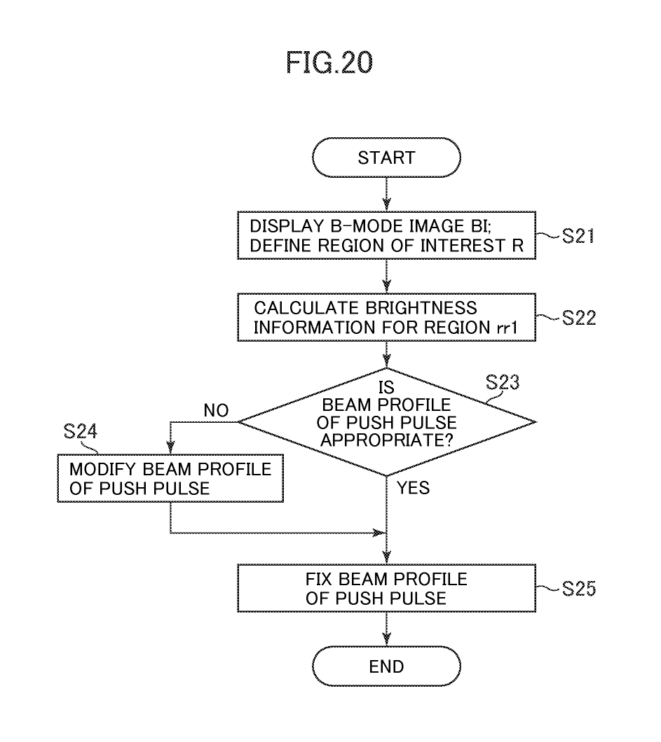

Next, a fourth variation will be described. In the present variation, it is assumed that the push pulses are transmitted to the same position. Fixing of the push pulses in the present variation will be described based on a flow chart in FIG. 20.

First, at Step 21, a B-mode image BI is displayed and a region of interest R is defined, as in Steps S1, S11 described earlier. Next, at Step S22, the evaluating section 44 calculates brightness information for a region rr in the B-mode image BI (see FIG. 13). The brightness information may be the mean brightness for the region rr or the degree of dispersion of brightness in the region rr.

Next, at Step S23, the evaluating section 44 evaluates whether degradation of image quality of an elasticity image by the inhibiting factor can be restrained or not by deciding whether the brightness information for the region rr meets predefined criteria or not to evaluate whether the beam profile of the push pulse corresponding to the region rr is appropriate or not. The decision by the evaluating section 44 whether the brightness information for the region rr meets predefined criteria or not is similar to that described earlier, which is based on the mean brightness Br or the degree of dispersion D for the region rr.

In the present variation, when Br>Brth, the evaluating section 44 decides that the beam profile of the push pulse is appropriate (“YES” at Step S23). On the other hand, when Br<=Brth, the evaluating section 44 decides that the beam profile of the push pulse is not appropriate (“NO” at Step S23). Alternatively, when D<Dth, the evaluating section 44 decides that the beam profile of the push pulse is appropriate. On the other hand, when D>=Dth, the evaluating section 44 decides that the beam profile of the push pulse is not appropriate.

The beam profile of the push pulse is, for example, a width of a transmission aperture for a push pulse. When the beam profile is decided not to be appropriate at Step S23, the flow goes to processing at Step S24. At Step S24, the control section 8 modifies the beam profile of the push pulse to an appropriate one. In particular, the beam profile is modified to such one that propagation of the push pulse by the inhibiting factor would not be intercepted. Here, the width of the transmission aperture for a push pulse is modified.

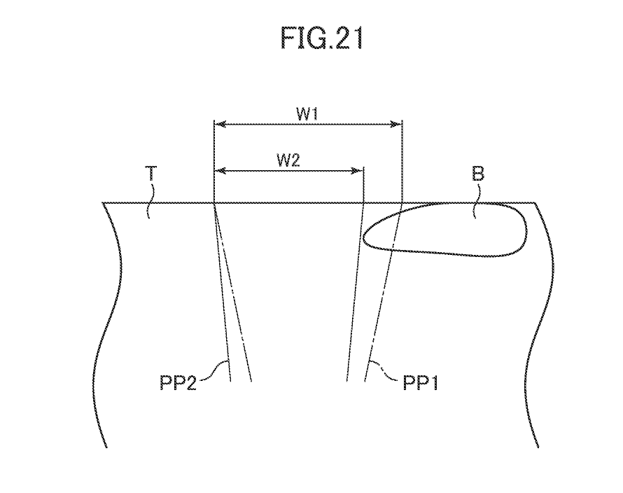

For example, as shown in FIG. 21, a width W1 of the transmission aperture for the push pulse PP1 indicated by a dot-dash line is reduced to a width W2 of the transmission aperture for the push pulse PP2 indicated by a solid line. The push pulse PP2 has a beam profile such that it avoids the bone B in the biological tissue T.

It should be noted that the width of the transmission aperture is reduced by reducing the number of ultrasonic vibrators for use in transmission of a push pulse.

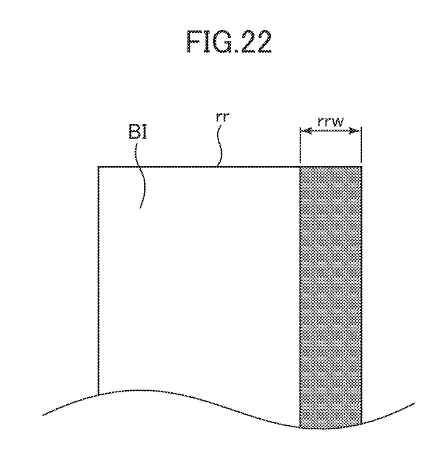

For example, as shown in FIG. 22, the width W2 of the transmission aperture may be determined based on a width rrw of a portion in which the brightness of the B-mode image BI is lower than a certain value in the region rr.

In case that the beam profile is decided to be appropriate at Step S23 (“YES” at Step S23) and in case that the beam profile is modified at Step S24, the flow goes to processing at Step S25. At Step S25, the beam profile decided to be appropriate at Step S23 or the beam profile modified at Step S24 is fixed as beam profile of a push pulse to be transmitted to the subject. The push pulse in the fixed beam profile is then transmitted and an elasticity image is displayed.

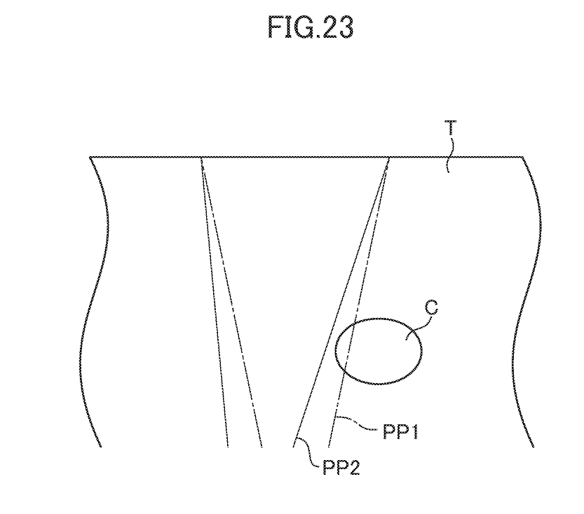

At Step S23 described above, a decision may be made as to whether the beam direction of a push pulse (acoustic line direction), in place of the beam profile of a push pulse, is appropriate or not. In this case, the control section 8 modifies the beam direction of a push pulse to an appropriate one at Step S24. Here, the beam direction is modified to such one that propagation of the push pulse by the inhibiting factor would not be intercepted. For example, as shown in FIG. 23, the beam direction of the push pulse PP1 indicated by a dot-dash line is modified to that of the push pulse PP2 indicated by a solid line. The push pulse PP2 has a beam direction such that it avoids a cyst in the biological tissue T.

Alternatively, at Step S23, a decision may be made as to whether both the beam profile and beam direction of a push pulse are appropriate or not. In this case, both the beam profile and beam direction of a push pulse are modified to appropriate ones at Step S24.

While the present invention has been described with reference to the embodiments, it will be easily recognized that the present invention may be practiced with several modifications without departing from the spirit and scope thereof. For example, in the second embodiment, instead of comparing the mean brightness Br for the region rr with the threshold Brth and deciding whether Br>Brth or not, the evaluating section 44 may compare the mean brightness Br for the region rr with thresholds Brth1, Brth2 (Brth1<Brth2) and decide whether Brth1<Br<Brth2 or not. In this case, when Brth1<Br<Brth2, degradation of image quality of the elasticity image by the inhibiting factor may be restrained. The threshold Brth1 is set to such a value that degradation of image quality of an elasticity image by the inhibiting factor probably cannot be restrained because of a low brightness of the region rr. The threshold Brth2 is set to such a value that degradation of image quality of an elasticity image by the inhibiting factor probably cannot be restrained because of a high brightness of the region rr. It should be noted that the surface of a bone has a higher brightness in a B-mode image.

Moreover, in the second embodiment, instead of comparing the degree of dispersion D in the region rr with a threshold Dth and deciding whether D<Dth or not, the evaluating section 44 may decide whether Dth1<D<Dth2 or not. In this case, when Dth1<D<Dth2, degradation of image quality of the elasticity image by the inhibiting factor may be restrained. For example, in case that the region rr has uniformly or mostly low brightness, the degree of dispersion lowers. Accordingly, Dthl is set to such a value that degradation of image quality of an elasticity image by the inhibiting factor probably cannot be restrained because an area having a low brightness is present in the region rr. Dth2 may be the same as Dth described earlier.

Furthermore, while the region of interest R is an example of the portion for which elasticity is to be measured in the present invention, the portion for which elasticity is to be measured in the present invention may be a point.

This written description uses examples to disclose the invention, including the preferred embodiments, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An ultrasonic diagnostic apparatus comprising:
a processor configured to:
control transmission of an ultrasonic push pulse to biological tissue in a subject, transmission of a plurality of ultrasonic detecting pulses for detecting shear waves generated in said biological tissue by said push pulse, and transmission of ultrasound for producing a B-mode image for said biological tissue,
calculate a measurement value regarding elasticity of said biological tissue in a portion for which elasticity is to be measured in said biological tissue based on echo signals of said ultrasonic detecting pulses, and
evaluate an impact of an inhibiting factor intercepting propagation of said shear waves toward said portion for which elasticity is to be measured on image quality of an elasticity image based on data indicating brightness for a predefined region in said B-mode image; and
an image display configured to display said B-mode image and said elasticity image,
wherein said push pulse is transmitted to such a position that degradation of image quality of said elasticity image by said inhibiting factor is restrained based on said evaluation.

2. The ultrasonic diagnosis apparatus according to claim 1, wherein:
a plurality of said predefined regions are defined at positions each corresponding to a transmission position to which each respective one of a plurality of said push pulses is to be transmitted,
said evaluation comprises comparing brightness information among the plurality of said predefined regions, and choosing a transmission position to which a push pulse is to be transmitted corresponding to one of the plurality of said predefined regions in which degradation of image quality of said elasticity image by said inhibiting factor may be restrained more, and
said push pulse is transmitted to the transmission position chosen by said evaluation.

3. The ultrasonic diagnosis apparatus as recited in claim 2, wherein:
said brightness information for said predefined region is a mean brightness for said predefined region, and
said evaluation comprises comparing the mean brightness among the plurality of said predefined regions and choosing a transmission position to which a push pulse is to be transmitted corresponding to a predefined region having the highest mean brightness.

4. The ultrasonic diagnosis apparatus as recited in claim 2, wherein:
said brightness information for said predefined region is a degree of dispersion of brightness in said predefined region, and
said evaluation comprises comparing said degree of dispersion among the plurality of said predefined regions and choosing a transmission position to which a push pulse is to be transmitted corresponding to a predefined region having the lowest degree of dispersion.

5. The ultrasonic diagnosis apparatus as recited in claim 1, wherein:
said predefined region is defined at a position corresponding to said transmission position to which a push pulse is to be transmitted,
said evaluation comprises deciding whether said brightness information for said predefined region meets predefined criteria or not to evaluate whether or not a transmission position to which a push pulse is to be transmitted corresponding to said predefined region is an appropriate one for restraining degradation of image quality of said elasticity image by said inhibiting factor, and
said push pulse is transmitted to a transmission position evaluated to be appropriate by said evaluation.

6. The ultrasonic diagnosis apparatus as recited in claim 5, wherein:
said brightness information for said predefined region is a mean brightness for said predefined region, and
said evaluation comprises comparing a mean brightness for said predefined region with a threshold and deciding whether said predefined criteria is met or not to evaluate whether the transmission position to which a push pulse is to be transmitted corresponding to said predefined region is appropriate or not.

7. The ultrasonic diagnosis apparatus as recited in claim 5, wherein:
said brightness information for said predefined region is a degree of dispersion of brightness in said predefined region, and
said evaluation comprises comparing a degree of dispersion in said predefined region with a threshold and deciding whether said predefined criteria is met or not to evaluate whether the transmission position to which a push pulse is to be transmitted corresponding to said predefined region is appropriate or not.

8. The ultrasonic diagnosis apparatus as recited in claim 1, wherein:
said predefined region is defined at a position corresponding to said transmission position to which a push pulse is to be transmitted,
said evaluation comprises detecting movement of a portion corresponding to said predefined region in said biological tissue based on data indicating brightness for said predefined region in said B-mode image to evaluate whether or not a transmission position to which a push pulse is to be transmitted corresponding to said predefined region is an appropriate one for restraining degradation of image quality of said elasticity image by said inhibiting factor, and
said push pulse is transmitted to a transmission position evaluated to be appropriate by said evaluation.

9. The ultrasonic diagnosis apparatus as recited in claim 1, wherein:
said predefined region is defined at a position corresponding to said transmission position to which a push pulse is to be transmitted,
said evaluation comprises deciding whether said brightness information for said predefined region meets predefined criteria or not to evaluate whether or not a beam profile and/or a beam direction for a push pulse corresponding to said predefined region is an appropriate one for restraining degradation of image quality of said elasticity image by said inhibiting factor, and
said push pulse is transmitted in a beam profile and/or a beam direction evaluated to be appropriate by said evaluation.

10. The ultrasonic diagnosis apparatus as recited in claim 9, wherein:
said brightness information for said predefined region is a mean brightness for said predefined region, and
said evaluation comprises comparing a mean brightness for said predefined region with a threshold and deciding whether said predefined criteria is met or not to evaluate whether the beam profile and/or beam direction of a push pulse corresponding to said predefined region is appropriate or not.

11. The ultrasonic diagnosis apparatus as recited in claim 9, wherein:
said brightness information for said predefined region is a degree of dispersion of brightness in said predefined region, and
said evaluation comprises comparing said degree of dispersion in said predefined region with a threshold and deciding said predefined criteria is met or not to evaluate whether the beam profile and/or beam direction of a push pulse corresponding to said predefined region is appropriate or not.

12. The ultrasonic diagnosis apparatus as recited in claim 9, wherein said beam profile is a width of a transmission aperture for said push pulse.

13. The ultrasonic diagnosis apparatus as recited in claim 1, wherein said predefined region includes an area to which said push pulse is to be transmitted.

14. The ultrasonic diagnosis apparatus as recited in claim 1, wherein said predefined region includes an area through which said shear waves propagate toward said portion for which elasticity is to be measured.

15. An ultrasonic diagnosis apparatus comprising:
a transmission controller configured to control transmission of an ultrasonic push pulse to biological tissue in a subject, transmission of a plurality of ultrasonic detecting pulses for detecting shear waves generated in said biological tissue by said push pulse, and transmission of ultrasound for producing a B-mode image for said biological tissue;
a measurement-value calculator configured to calculate a measurement value regarding elasticity of said biological tissue in a portion for which elasticity is to be measured in said biological tissue based on echo signals of said ultrasonic detecting pulses;
an evaluator configured to evaluate an impact of an inhibiting factor intercepting propagation of said shear waves toward said portion for which elasticity is to be measured on image quality of an elasticity image based on data indicating brightness for a predefined region in said B-mode image; and
an image display configured to display said B-mode image and said elasticity image,
wherein said transmission controller transmits said push pulse to such a position that degradation of image quality of said elasticity image by said inhibiting factor is restrained based on an evaluation by said evaluator.

16. A method for ultrasonic imaging, the method comprising:
controlling transmission of an ultrasonic push pulse to biological tissue in a subject, transmission of a plurality of ultrasonic detecting pulses for detecting shear waves generated in said biological tissue by said push pulse, and transmission of ultrasound for producing a B-mode image for said biological tissue;
calculating a measurement value regarding elasticity of said biological tissue in a portion for which elasticity is to be measured in said biological tissue based on echo signals of said ultrasonic detecting pulses; and
evaluating an impact of an inhibiting factor intercepting propagation of said shear waves toward said portion for which elasticity is to be measured on image quality of an elasticity image based on data indicating brightness for a predefined region in said B-mode image,
wherein said push pulse is transmitted to such a position that degradation of image quality of said elasticity image by said inhibiting factor is restrained based on said evaluation.
